(19) United States
(12) Patent Application Publication
(10) Pub. No.: US 2007/0166404 A1
(43) Pub. Date: Jul. 19, 2007

(54) Composition and method for supporting cancer treatments

(76) Inventors: Hsun-Lang Chang, Taoyuan (TW); Wu-Chang Chuang, Taipei (TW); Wei-Ying Kuo, Taipei County (TW); Guang-Teuu Shane, Taipei (TW); Hung-Sheng Chang, Taipei (TW)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/552,029

(22) PCT Filed: Apr. 3, 2003
(86) PCT No.: PCT/SG03/00071

§ 371 Date: Oct. 18, 2006

(51) Int. Cl. A61K 36/489

(52) U.S. Cl. 424/725; 424/773; 424/757

(57) ABSTRACT

The present invention relates to a novel composition comprising geranium oil and extracts from the roots of the plants of the genus Sophora, preferably Sophora tonkinesis. Said composition can be administered to mammalian animals undergoing cancer treatments, such as chemotherapy and radiation therapy, that would induce the side effect of bone marrow suppression. The administration can be made before, during and or after the cancer treatment.

### BACKGROUND OF THE INVENTION

1. Field of Invention

This invention relates generally to a novel composition to be used as a supporting composition in cancer treatments and more particularly to an herbal composition and method of using said herbal composition together with chemotherapy or radiation therapy (or both) in the treatment of cancer.

2. Description of Related Art

Normal cells grow and divide in an orderly and controlled manner. Cancer is a disease where cells become abnormal (cancerous cells) and begin to multiply without control to develop into an extra mass of tissue called a tumor. These cancerous cells can invade nearby tissues and spread through the blood stream and lymphatic system to other parts of the body.

Currently, the four primary types of cancer treatments are immunotherapy, surgery, radiation therapy, and chemotherapy. These cancer treatments may be applied alone or in conjunction with one another. Thus a cancer patient may undergo one or more treatments at a time. A single treatment would span a predefined period of time with therapies delivered at various timed intervals. Immunotherapy, also known as biological therapy or biological response modifier (BRM) therapy, tries to stimulate or restore the ability of the immune system to fight the disease. It is also used to lessen immune system related side effects that may be caused by some cancer treatments. Surgery seeks to directly remove the tumor from the body.

Radiation therapy, also known as radiotherapy, uses high-energy radiation from x-rays, gamma rays, neutrons, and other sources to kill cancer cells and shrink tumors by damaging the cells' genetic material. While cancerous cells are damaged permanently and eventually die, some normal cells that are damaged in radiation therapy are also unable to repair themselves. Side effects that can occur during radiation therapy include skin irritation and hair loss in the area being treated and damage to the bone marrow.

Chemotherapy uses cytotoxic drugs, alone or in combination, to destroy cancer cells. Just as in radiation therapy, cancer cells can be damaged and eventually die. But only some healthy cells affected in the process can repair themselves after the chemotherapy. Cytotoxic drugs work by interfering with the ability of a growing cell to divide and reproduce itself. Thus, in addition to cancerous cells, other normal fast-dividing growing cells can also be affected. There can be an effect on blood cells forming in the bone marrow causing bone marrow suppression. There can also be an effect on cells in the digestive tract, in the lining of the mouth and in the reproductive system causing diarrhea and mouth soreness, and an effect on hair follicles causing hair loss.

Bone marrow suppression is one of the many side effects of chemotherapy and radiation therapy. It results in reduced blood cell production, including red blood cells, white blood cells, and platelets. Consequently, the patient will experience tiredness, from anemia, become more susceptible to infections, from leukopenia, and bruise easily and bleed more when getting a cut, from thrombocytopenia.

Drugs are used to counter the bone marrow suppression side effect. Epogen (Epoietin alpha) has been used to counter the side effect of anemia in cancer chemotherapy, and WinRho SD has been used to counter the side effect of thrombocytopenia.

Many of the treatments developed to coordinate with chemotherapy and radiation therapy to counter the side effect of leukopenia act on specific types of white blood cells, i.e. granulocytes, monocytes, and lymphocytes. Neupogen is a recombinant human granulocyte colony-stimulating factor (G-CSF) that stimulates the growth of neutrophils. Leukine is a recombinant human granulocyte-macrophage colony-stimulating factor that stimulates the production of neutrophils and macrophages. In animal laboratories and clinical trials, various interleukins, secreted by T-lymphocytes, have been used to stimulate productions of various white blood cells in the course of or after chemotherapy. interleukin 1 (IL-1) is responsible for B-cell and T-cell proliferation, interleukin 2 (IL-2) is responsible for the proliferation, growth, and activation of B-cells and T-cells, interleukin 15 (15) appears to be required for natural killer cells (NK cells) and CD8+ T-cells.

Herbs have also been found to have the activity of countering leukopenia side effect. For example, injection of extracts from Sophora flavescenes roots has been reported to have reduced leukopenia side effect of chemotherapy and radiation therapy. Also, injection of Uncaria tomentosa water extracts in rats experiencing leukopenia from chemotherapy led to an increase in white blood cells.

### BRIEF SUMMARY OF THE INVENTION

The present invention is directed to a novel composition and method of using the novel composition in cancer treatments, preferably to reduce the bone marrow suppression side effect of such treatments. The novel composition is made of geranium oil and extractions from the root of Sophora plants, preferably Sophora tonkinesis, also known as Sophora subprostrata, (referred to herein as Sophora tonkinesis). The above “geranium oil” and “extractions from the root of Sophora plants” preferably refer to the main ingredients directly extracted from the oil and root respectively, but also includes main ingredients that are chemically synthesized or otherwise provided. The herbal composition can take on many forms e.g., powders, oil capsules, tablets, pills, liquid, syrup or pastes. The herbal composition can made into and ingested as a food additive, dietary supplement, health food, decoction soup, or any other edible form. The herbal composition can be administered via various routes, i.e. oral, intravenous, or intraperitoneal, in specific dosages to mammalian animals undergoing chemotherapy or radiation therapy. For administration, the composition can be obtained by preparation, purchase, or any other means so one is in possession of the composition and administered before, during and after the cancer treatments.

### BRIEF DESCRIPTION OF THE DRAWINGS

FIG. 1 shows the compounds identified and their relative contents in the geranium oil produced in Kunming, China by the methods of gas chromatography/mass spectroscopy.

FIG. 2 shows the result of pharmcokinetics study of intravenous injection of matrine and matrine with and addition of geranium oil.

FIG. 3 shows the result of pharmcokinetics study of intravenous injection of oxymatrine and oxymatrine with the addition of geranium oil.

### DETAILED DESCRIPTION OF THE INVENTION

The present invention relates to a novel composition comprising geranium oil and extractions from the root of Sophora plants, preferably Sophora tonkinesis, and method of using the novel composition as a supporting drug or supplement in cancer treatments, preferably to reduce the bone marrow suppression side effect occurring in most of such treatments.

1. Geranium Oil

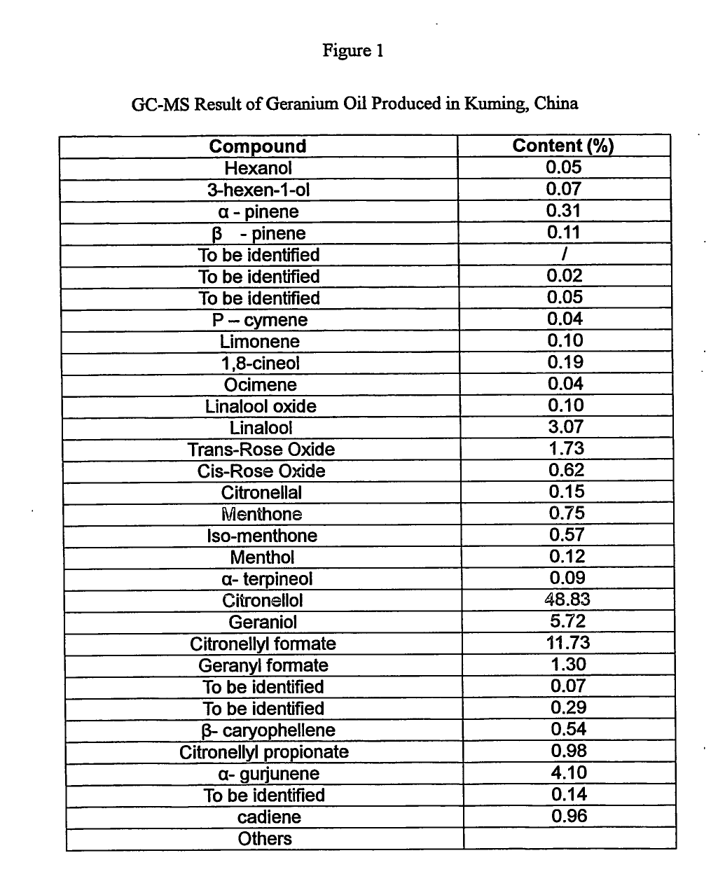

Geranium oil may be collected from steam distillation of the stem and leaves of the plant of division Magnoliophyta, class Magnoliopsida, order Geraniales, family Geraniaceae, and genus Pelargonium. Pelargoniums are native to South Africa and there are more than one hundred species in existence today, including hybridized garden species. Pelargoniums are now grown, and geranium oil is now produced, mainly in Algeria, Egypt, Morocco, Bourbon, China, and Australia. The present invention preferably uses geranium oil extracted from Pelargonium graveolens or Pelargonium roseum and Pelargonium terebinthinceum grown in Kunming City of the Yunan Province in China. Agas chromatography/mass spectroscopy (GC-MS) result of the geranium oil produced in Kunming shows the constituent compounds and their relative contents (see FIG. 1). The generally known main constituents of geranium oil are citronellol, geraniol, geranyl formate, citronellyl formate, linalool, trans-rose oxide, and cis-rose oxide.

Certain specifications of geranium oil are set out in the National Standard of the People's Republic of China—GB 11959-89 which is incorporated herein by reference in their entirety, including any drawings. It adopts the same international standard of ISO 4731:1978 Oil of Geranium (Geranium Oil Standard). The Geranium Oil Standard specifies the outward characteristics of geranium oil, i.e. the geranium oil takes on a clear oil liquid form of a yellow greenish or amber color and has a distinct aroma. The same standard also specifies a relative density of 0.881-0.900 g/cm³, an optical rotation of −60° to −14°, and a refractive index of 1.459-1.466 for geranium oil.

2. Sophora tonkinesis

The root of Sophora tonkinesis takes on a long curved tublar form with branches and is typically about 0.3-1.5 centimeters in diameter. The root is hardened and difficult to break. Its surface color ranges from grayish brown to suntan brown with longitudinal wrinkles and holes. The root has a bean scent and is extremely bitter. It is grown mainly in parts of China, i.e. the Guangdong province, Guangxi province, Guizhou province, Yunan province, and Jiangxi province.

The root contains 0.93% of alkaloids, of which 0.52% is matrine and 0.35% is oxymatrine. The other alkaloids identified in the root of Sophora tonkinesis are anagyrine, methylcytisine, cytosine, sophocarpine, sophocarpine N-oxide, sophoramine, and sophoranol. The flavonic compounds identified in the root are sophoranone, sophoradin, sophoranochromene, sophoradochromene, pterocarpine, genistein, maackian, trifolirhizin, sitosterol, lu-peol, and a group of alkyl alcohol ester.

The principal alkaloid constituents of Sophora tonkinesis are also found in Sophora alopecuioides, Sophora moorcroftiana, and Euchresta strigillosa.

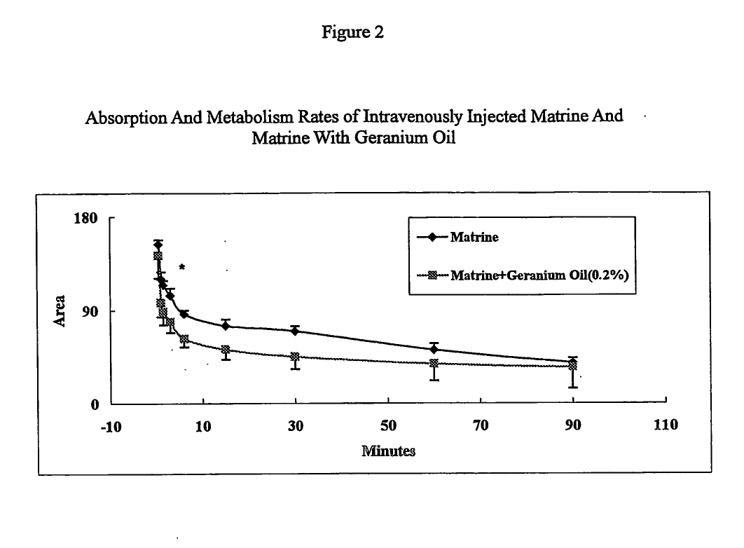

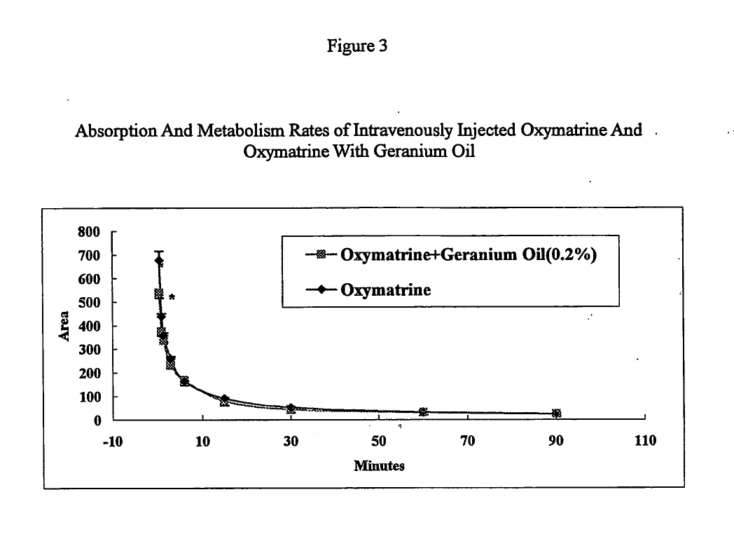

Result of pharmcokinetics study shows that in intravenous injections, the addition of geranium oil to matrine or oxymatrine will increase the absorption and metabolism of the respective compound (please see FIG. 2 and FIG. 3 for the changes in HPLC peak areas of matrine and matrine+geranium oil as time progresses). Furthermore, the composition of the present invention, containing oxymatrine, can also be taken orally to increase white blood cells. This is contrary to previously published data of animal experiments and clinical trials indicating that oxymatrine, when taken orally does not show any effect on increasing white blood cells, has to be injected through the muscles to increase white blood cells.

3. Composition

The composition can be formed into powders (composition powders) through the following steps. First, geranium oil and the root of Sophora tonkinesis are prepared separately. β-cyclodextrin is added to geranium oil to prevent evaporation, and excipients are added subsequently to form geranium oil powders. The geranium oil and the excipients are about 31% and 62% by weight, respectively, of the geranium oil powders. Next, the root of Sophora tonkinesis is cut into thin pieces and then grounded. About 250 grams of the grounded Sophora tonkinesis root is mixed with 3000 ml of water, about 12 times the weight of the grounded root. The mixture is then boiled in a steam distillation bottle to heat and reflux for about 1 hour. Afterwards; the scum on the surface of the liquid is removed, and the liquid is filtered through a 100 mesh screen. The filtered liquid is then concentrated and about 66 grams of solid extracts of Sophora tonkinesis is obtained. Excipients are added to the solid extractions to form Sophora tonkinesis root powders. The Sophora tonkinesis extractions and the excipients are about 60% and 40% by weight, respectively, of the Sophora tonkinesis powders. Subsequently, the geranium oil powders and the Sophora tonkinesis root powders are mixed together with additional excipients to form the composition of the present invention into powder forms, wherein the geranium oil powders, Sophora tonkinesis root powders, and the excipients are about 55.94%, 0.958%, and 43.102% by weight, respectively, of the composition powders. The weight ratio of geranium oil and extractions of Sophora tonkinesis within the composition are about 30:1. The excipients to be used in the process to form powders can be starch, sugar spheres, fructose, sorbital crystalline etc. and those commonly used by one skilled in the art.

Alternatively, the geranium oil powders and the Sophora tonkinesis root powders can be mixed with glycerine and gelatin to form capsules. The composition can also be made into dietary supplement, health food (functional food), and food additives. One can also decoct the Pelargonium plant and Sophora roots to obtain a liquid form of the composition for direct oral intake as a medicine soup or for making into syrup or other forms of liquid composition. Sophora roots the Pelargonium plant can also be taken orally, in an edible form, separately at a timed interval.

### EXAMPLE

Composition powders were administered orally to immunologically normal mice that were also given the 5-Fluorouracil (5-Fu) drug intraperitoneally.

The test substance, i.e. the composition powders, was prepared by dissolving the content in PBS.

Animals tested are 12 male BALB/c mice of 6-7 weeks old, weighing 22±2 grams, provided by Taiwan National University Medical Center Laboratory Animal Center. The animals are divided into two groups of 6 mice. Laboratory mice feeds manufactured by Purina (PMI5001) were used. Double-distilled water was provided for drinking. Laboratory mice wooden beddings manufactured by Beta Chip were used and changed 2-3 times weekly. Each group of 6 mice was kept in a feeding box of 29.2×19×12.7 (cm). Micro-Isolator™ VCL Rack Housing System 70084A was used. Temperature and humidity were kept at 23±2° C. and 6±10% respectively. The mice were given twelve hours of light and twelve hours of darkness.

Doses of 21 mg and 7 mg of test substance dissolved in PBS were administered to the two groups of test animals respectively, in a feeding volume of 0.2 ml/mouse. The test substance were adnistered orally to the test animals the day after a single dose of the chemotherapeutic agent 5-Fu (135 mg/kg, IP) was given and then once daily for the next nine (10 doses total) and thirteen consecutive days (14 doses in total) for the first and second group of mice respectively. On day 10, the first group of mice was sacrificed by anesthetizing with CO2 and taking the blood from the heart to determine the cell counts of erythrocytes (RBC), platelets (PLT), total leukocytes (WBC), and differential leukocytes counts: lymphocytes (LY), monocytes (MO), and granulocytes (GR). On day 14, the second group of mice was sacrificed in the same manner to determine the same blood cell counts. The control employed in the experiment were normal mice without any injections.

As shown in the table below, 7 mg/mouse of test substance had the significant effect of increasing the number of red blood cells (RBC) and preventing the reduction of the number of WBC, LY, MO, and GR in mice injected with 5-Fu. The effect was more pronounced with the dosage of 7 mg/mouse. On day 10, the average WBC count of normal mice was 6.94±1.647×10³/μl, and the mice treated with 5-Fu had an average WBC count of4.17±0.677×10³/μl. On the other hand, mice treated with 7 mg/mouse of test substance and 5-Fu had an average WBC count of 6.24±1.924×10³/μl, showing only 25% of the bone marrow suppression effect of 5-Fu. Differential leukocyte count showed that the suppression effect with respect to lymphocytes in test animals treated with 7 mg/mous of test substance and 5-Fu was only 12% of that of the test animals treated with 5-Fu only. With respect to monocytes, the suppression effect in test animals treated with 7 mg/mouse of test substance and 5-Fu was only 21% ofthat oftest animals treated with 5-Fu. With respect to granulocytes, the suppression effect in test animals treated with 7 mg/mouse of test substance and 5-Fu was 46% of that of test animals treated with 5-Fu. On day 14, the total leukocyte and differential leukocyte counts of mice treated with 7 mg/mouse of test substance and 5-Fu continued to increase to a higher level than that of mice treated with 5-Fu only.

Effect of ^{┌} geranium oil + Sophora tonkinesis extractions┘ on the side effects
of reduction in blood cell counts caused by 5-Fu-

| | | | 21 mg/mouse S. | 7 mg/mouse S. |
|---|---|---|---|---|
| | Normal | 5-Fu | tonkinesis/5-Fu | tonkinesis/5-Fu |
| Day 10 | | | | |
| RBC (10⁶/μl) | 9.09 ± 0.137 | 7.86 ± 0.171 | 7.66 ± 0.316 | 8.52 ± 0.627* |
| PLT (10³/μl) | 990 ± 65.7 | 2828 ± 632.4 | 2441 ± 441.4 | 2099 ± 731.5 |
| WBC (10³/μl) | 6.94 ± 1.647 | 4.17 ± 0.677 | 4.63 ± 0.772 | 6.24 ± 1.924* |
| LY (10³/μl) | 5.30 ± 1.369 | 3.66 ± 0.648 | 4.15 ± 0.538 | 5.10 ± 1.261* |
| MO (10³/μl) | 0.39 ± 0.035 | 0.25 ± 0.046 | 0.26 ± 0.154 | 0.36 ± 0.131* |
| GR (10³/μl) | 1.24 ± 0.284 | 0.25 ± 0.050 | 0.22 ± 0.104 | 0.78 ± 0.559* |
| Day 14 | | | | |
| RBC (10⁶/μl) | 9.76 ± 0.269 | 8.09 ± 0.331 | 8.19 ± 0.160 | 8.23 ± 0.326 |
| PLT (10³/μl) | 985 ± 216.5 | 2219 ± 750.2 | 2461 ± 195.4 | 2309 ± 687.5 |
| WBC (10³/μl) | 803 ± 1.408 | 7.98 ± 1.575 | 7.70 ± 0.599 | 8.48 ± 2.052 |
| LY (10³/μl) | 6.53 ± 1.470 | 5.75 ± 0.880 | 6.10 ± 0.397 | 6.56 ± 1.591 |
| MO (10³/μl) | 0.35 ± 0.092 | 0.59 ± 0.316 | 0.39 ± 0.124 | 0.44 ± 0.140 |
| GR (10³/μl) | 1.15 ± 0.243 | 1.65 ± 0.756 | 1.21 ± 0.353 | 1.47 ± 0.560 |

1. Results are expressed in mean ± standard deviation (mean ± SD).
2. The experimental group and the 5-Fu group are compared using Dunnett's t-test, “*” means p<0.05, and “**” means p<0.01, and “***” means p<0.001.

The weight of mice treated with 7 mg/mouse and 21 mg/mouse decreased slightly, as the days progresses, as compared to the normal mice.

Effect of geranium oil + Sophora tonkinesis extractions on the side effects
of weight change caused by 5-Fu

| | Day -2 | Day 0 | Day 6 | Day 10 | Day 14 |
|---|---|---|---|---|---|
| Normal control | 20.3 ± 1.90 | 21.6 ± 1.85 | 23.1 ± 1.94^{a} | 24.0 ± 1.89^{a} | 24.6 ± 2.21 |
| | (n = 18) | (n = 18) | (n = 18) | (n = 12) | (n = 6) |
| 5-Fu | 20.7 ± 0.90 | 22.0 ± 0.93 | 21.9 ± 1.20^{ab} | 22.3 ± 1.53^{b} | 23.5 ± 0.78 |
| | (n = 12) | (n = 12) | (n = 12) | (n = 12) | (n = 6) |
| G-CSF/5-Fu | 20.5 ± 1.76 | 22.0 ± 1.84 | 22.0 ± 1.84^{ab} | 22.5 ± 1.78^{ab} | 24.0 ± 1.64 |
| | (n = 12) | (n = 12) | (n = 12) | (n = 12) | (n = 6) |
| 21 mg/mouse | 19.6 ± 1.42 | 21.2 ± 1.38 | 21.4 ± 1.71^{b} | 21.4 ± 1.80^{b} | 22.3 ± 1.57 |
| S. | (n = 12) | (n = 12) | (n = 12) | (n = 12) | (n = 6) |
| tonkinesis/5-Fu | | | | | |
| 7 mg/mouse | 19.8 ± 1.50 | 21.4 ± 1.97 | 22.2 ± 1.67^{ab} | 22.6 ± 1.68^{ab} | 23.1 ± 3.04 |
| S. | (n = 12) | (n = 12) | (n = 12) | (n = 12) | (n = 6) |
| tonkinesis/5-Fu | | | | | |

1. Results are expressed in mean ± standard deviation (mean ± SD).
2. At the same moment in time, Duncan's statistical analysis is used among the groups. Different alphabets stands for significant differences (p<0.05).

In comparison with another type of treatment relating to the reduction of bone marrow suppression using G-CSF, the bone marrow suppression effect was not as significantly reduced as that ofthe tested novel composition ofthe present invention. On day 10, the normal mice's average WBC count was 6.94±1.647×10³/μl and the mice treated with 5-Fu had an average WBC count of 4.17±0.677×10³ μl. On the other hand, mice treated with 135 μg/mouse of G-CSP and 5-Fu had an average WBC count of 5.46±2.338×10³/μl, showing 51% of the bone marrow suppression effect of 5-Fu. Differential leukocyte count showed that the suppression effect with respect to lymphocytes in test animals treated with 135 μg/mouse of G-CSF and 5-Fu was only 18% of that of the test animals treated with 5-Fu only. With respect to monocytes, the suppression effect in test animals treated with 135 μg/mouse of G-CSF and 5-Fu was only 7% of that of test animals treated with 5-Fu. With respect to granulocytes, the suppression effect in test animals treated with 135 μg/mouse of G-CSF and 5-Fu was 54% of that of test animals treated with 5-Fu. On day 14, only the total leukocyte count and differential leukocyte count, with respect to lymphocytes of mice treated with 135 μg/mouse of G-CSF and 5-Fu, continued to increase to a higher level than that of mice treated with 5-Fu only.

Effect of G-CSF on the reduction of 5-Fu's side effect-change
in blood cell counts

| | | | 135 μg/mouse |
|---|---|---|---|
| | Normal | 5-Fu | C-GSF/5-Fu |
| Day 10 | | | |
| RBC (10⁶/μl) | 9.09 ± 0.137 | 7.86 ± 0.171 | 7.82 ± 0.424 |
| PLT (10³/μl) | 990 ± 65.7 | 2828 ± 632.4 | 2303 ± 491.7 |
| WBC (10³/μl) | 6.94 ± 1.647 | 4.17 ± 0.677 | 5.46 ± 2.338 |
| LY (10³/μl) | 5.30 ± 1.369 | 3.66 ± 0.648 | 5.01 ± 1.372* |
| MO (10³/μl) | 0.39 ± 0.035 | 0.25 ± 0.046 | 0.38 ± 0.141* |
| GR (10³/μl) | 1.24 ± 0.284 | 0.25 ± 0.050 | 0.71 ± 0.268** |
| Day 14 | | | |
| RBC (10⁶/μl) | 9.76 ± 0.269 | 8.09 ± 0.331 | 8.02 ± 0.340 |
| PLT (10³/μl) | 985 ± 216.5 | 2219 ± 750.2 | 2105 ± 378.1 |
| WBC (10³/μl) | 8.03 ± 1.408 | 7.98 ± 1.575 | 8.34 ± 1.454 |
| LY (10³/μl) | 6.53 ± 1.470 | 5.75 ± 0.880 | 6.21 ± 1.164 |
| MO (10³/μl) | 0.35 ± 0.092 | 0.59 ± 0.316 | 0.58 ± 0.266 |
| GR (10³/μl) | 1.15 ± 0.243 | 1.65 ± 0.756 | 1.64 ± 0.405 |

1. Results are expressed in mean ± standard deviation (mean ± SD).
2. The experimental group and the 5-Fu group are compared using Dunnett's t-test, “*” means p<0.05, and “**” means p<0.01, and “***” means p<0.001.

The weight of the mice shows no significant difference among the groups.

Effect of G-CSF on the reduction of 5-Fu's side effect-weight change

| | Day -2 | Day 0 | Day 6 | Day 10 | Day 14 |
|---|---|---|---|---|---|
| Normal control | 20.3 ± 1.90 | 21.6 ± 1.85 | 23.1 ± 1.94 | 24.0 ± 1.89^{a} | 24.6 ± 2.21 |
| | (n = 18) | (n = 18) | (n = 18) | (n = 12) | (n = 6) |
| 5-Fu | 20.7 ± 0.90 | 22.0 ± 0.93 | 21.9 ± 1.20 | 22.3 ± 1.53^{b} | 23.5 ± 0.78 |
| | (n = 12) | (n = 12) | (n = 12) | (n = 12) | (n = 6) |
| G-CSF/5-Fu | 20.5 ± 1.76 | 22.0 ± 1.84 | 22.0 ± 1.84 | 22.5 ± 178^{b} | 24.0 ± 1.64 |
| | (n = 12) | (n = 12) | (n = 12) | (n = 12) | (n = 6) |

1. Results are expressed in mean ± standard deviation (mean ± SD).
2. At the same moment in time, Duncan's statistical analysis is used among the groups. Different alphabets stands for significant differences (p<0.05).

The composition of Sophora tonkinesis and geranium oil does in fact significantly reduces the bone marrow suppression effect of 5-Fu and is performing better even than the G-CSF treatment. The ability of the composition of the present invention to reduce bone marrow suppression effect makes it a good candidate as a supporting drug or supplement to be used in cancer treatments that induce bone marrow suppression. In particular, the composition of the present invention may be used with chemotherapy and or radiation therapy to increase the leukocyte count. For example, the composition of the present invention may be used with 5-Fu, doxorubincin and other chemotherapeutic agents just as Neupogen is also used with 5-Fu as well as doxorubincin and many other types of chemotherapy to stimulate the growth of neutrophils whose number is originally reduced by chemotherapy.

Human dosages can be calculated based on the dosages used for mice in the experiment. In accordance with accepted clinical trial practice, mice dosages are divided by a factor of 10 in order to obtain suitable and safe dosages for human. A range of dosage is calculated based on the premise that the mice weighed about 20-25 grams. The calculation is shown as follows.
7 mg/mouse/day→7 mg/20 g/day×50→350 mg/kg/day÷10×60=2100 mg/60 kg/day
7 mg/mouse day→7 mg/25 g/day×50→280 mg/kg/day÷10×60=1680 mg/60 kg/day
21 mg/mouse/day→21 mg/20 g/day×50→1050 mg/kg/day÷10×60=6300 mg/60 kg/day
21 mg/mouse/day→21 mg/25 g/day×50→840 mg/kg/day÷10×60=5040 mg/60 kg/day

1. A composition comprising geranium oil and extractions from the root of Sophora tonkinesis.

2. The composition of claim 1, wherein said geranium oil and extractions from the root of Sophora tonkinesis have a weight ratio of about 30:1.

3. The composition of claim 1, wherein said composition takes on the form of a mixture of powders of geranium oil and powders of extracts from the root of Sophora tonkinesis.

4. The composition of claim 3, wherein said mixture further comprises excipients

5. The composition of claim 3, wherein said powders of geranium oil is about 55.94% of the mixture of powders.

6. The composition of claim 5, wherein said powders of geranium oil further comprises excipients.

7. The composition of claim 3, wherein said powders of extractions from the root of Sophora tonkinesis is about 0.958% of the mixture of powders.

8. The composition of claim 7, wherein said powders of extractions from the root of Sophora tonkinesis further comprises excipients.

9. The composition of claim 1, wherein said composition takes on the form of oil capsule, tablets, pills, liquid or pastes.

10. The composition of claim 1, wherein said geranium oil is extracted from one or more species of the genus Pelargonium.

11. The composition of claim 1, wherein said geranium oil is extracted from a plant of the genus Pelargonium and species graveolens.

12. The composition of claim 1, wherein said geranium oil is extracted from a plant of the genus Pelargonium and species roseum.

13. The composition of claim 1, wherein said geranium oil is extracted from a plant of the genus Pelargonium and species terebinthinceum.

14. A composition comprising citronellol, geraniol, geranyl formate, citronellyl formate, matrine, and oxymatrine.

15. A composition comprising citronellol, geraniol, geranyl formate, citronellyl formate, linalool, trans-rose oxide, cis-rose oxide, matrine, oxymatrine, and sophocarpine.

16. A composition comprising geranium oil, matrine, and oxymatrine.

17. A composition comprising extractions from the root of Sophora tonkinesis, citronellol, geraniol, citronellyl formate, and geranyl formate.

18. A composition comprising A and B wherein A is selected from a group consisting of hexanol, 3-hexen-1-ol, α-pinene, β-pinene, P-cymene, limonene, 1,8-cineol, ocimene, linallol oxide, linallol, trans-rose oxide, cis-rose oxide, citronellal, menthone, iso-methone, menthol, terpineol, citronellol, geraniol, citronellyl formate, geranyl formate, caryophellene, citronellyl propinoate, gurjunene, cadiene, and B is selected from a group consisting of matrine, oxymatrine, anagyrine, methylcytisine, cytosine, sophocarpine, sophocarpine N-oxide, sophoramine, sophoranol, sophoranone, sophoradin, sophoranochromene, sophoradochromene, pterocarpine, genistein, maackian, trifolirhizin, sitosterol, lu-peol, and alkyl alcohol ester.

19. The composition of claims 1, 9, 14, 15, 16, 17, or 18, wherein said composition further comprises a pharmaceutically acceptable solvent.

20. A method for administering a composition comprising the steps of:
(a) locating one or more mammalian animals being treated or to be treated with one or more cancer treatments;
(b) determining a route of administering said composition to said one or more mammalian animals;
(c) determining a form of said composition to be administered to said one or more mammalian animals;
(d) obtaining a therapeutically effective dosage of said composition wherein said composition comprises geranium oil and extractions from the root of Sophora tonkinesis;
(e) placing said composition in an apparatus for administration; and
(f) delivering said dosage of said composition to said one or more mammalian animals being treated or to be treated with said one more cancer treatments.

21. The method of claim 20, wherein said composition comprises the composition of claims 14, 15, 16, 17, or 18.

22. The method of claim 20, wherein said one or more mammalian animals are one or more humans.

23. The method of claim 20, wherein said one or more mammalian animals are one or more canines or monkeys.

24. The method of claim 20, wherein said one or more mammalian animals are non-rodents.

25. The method of claim 20, wherein said one or more mammalian animals are rodents.

26. The method of claim 25, wherein said rodents are mice, rats, rabbits, or hamsters.

27. The method of claim 20, wherein said one or more cancer treatments induce a bone marrow suppression side effect.

28. The method of claim 20, wherein said one or more cancer treatments induce a leukopenia side effect.

29. The method of claim 20, wherein said one or more cancer treatments involve the administration of one or more chemotherapeutic agents.

30. The method of claim 29, wherein said chemotherapeutic agent induces the bone marrow suppression side effect.

31. The method of claim 29, wherein said chemotherapeutic agent induces the leukopenia side effect.

32. The method of claim 29, wherein said chemotherapeutic agent is 5-Fluorouracil.

33. The method of claim 29, wherein said chemotherapeutic agent is doxorubincin.

34. The method of claim 20, wherein said one or more cancer treatments involve radiation therapy.

35. The method of claim 20, wherein said step of delivering said dosage of said composition is carried out by delivering said composition to said mammalian animals before they are being treated with said one or more cancer treatments.

36. The method of claim 20, wherein said step of delivering said dosage of said composition is carried out by delivering the composition to said mammalian animals before and after they are being treated with said one or more cancer treatments.

37. The method of claim 20, wherein said step of delivering said dosage of said composition is carried out by delivering said composition to said mammalian animals after they are being treated with said one or more cancer treatments.

38. The method of claim 20, wherein said step of delivering said dosage of said composition is carried out by delivering said composition to said mammalian animals during said one or more cancer treatments.

39. The method of claim 20, wherein said route of delivering said dosage of said composition is oral administration.

40. The method of claim 39, wherein said dosage is in a range between about 280 mg/kg/day and about 6300 mg/60 kg/day.

41. The method of claim 39, wherein said oral administration is carried out by administering powders comprising said composition to said one or more mammalian animals.

42. The method of claim 39, wherein said oral administration is carried out by administering one or more tablets comprising said composition to said one or more mammalian animals.

43. The method of claim 39, wherein said oral administration is carried out by administering one or more oil capsules comprising said composition to said one or more mammalian animals.

44. The method of claim 39, wherein said oral administration is carried out by administering one or more pills comprising said composition to said one or more mammalian animals.

45. The method of claim 39, wherein said oral administration is carried out by administering pastes comprising said composition to said one or more mammalian animals.

46. The method of claim 39, wherein said oral administration is carried out by administering food additives comprising said composition to said one or more mammalian animals.

47. The method of claim 39, wherein said oral administration is carried out by administering a dietary supplement comprising said composition to said one or more mammalian animals.

48. The method of claim 39, wherein said oral administration is carried out by administering health food comprising said composition to said one or more mammalian animals.

49. The method of claim 39, wherein said oral administration is carried out by administering a liquid comprising said composition to said one or more mammalian animals.

50. The method of claim 39, wherein said oral administration is carried out by administering a syrup comprising said composition to said one or more mammalian animals.

51. The method of claim 39, wherein said oral administration is carried out by administering decoction soup comprising said composition to said one or more mammalian animals.

52. The method of claim 39, wherein said oral administration is carried out by administering edible forms of Pelargonium plant and the root of Sophora tonkinesis.

53. The method of claim 39, wherein said oral administration is carried out by administering said geranium oil and powders of the root of Sophora tonkinesis.

54. The method of claim 39, wherein said oral administration is carried out by administering geranium oil and pastes made from the root of Sophora tonkinesis.

55. The method of claim 39, wherein said oral administration is carried out by administering geranium oil and soup mixture from decocting the root of Sophora tonkinesis.

56. The method of claim 39, wherein said oral administration is carried out by administering said extractions from the root of Sophora tonkinesis and soup mixture from decocting the Pelargonium plant.

57. The method of claim 39, 41, 42, 43, 44, 45, 46, 47, 48, 49, 50, 51, 52, 53, 54, 55 or 56, wherein said composition of extractions from the Pelargonium plant and the root of Sophora tonkinesis has a weight ratio of about 30:1.

58. The method of claim 20, wherein said route of administering said composition is carried out by administering intraperitoneally said composition to said one or more mammalian animals.

59. The method of claim 20, wherein said route of administration is carried out by administering intravenously said composition to said one or more mammalian animals.

60. A method for administering a composition comprising the steps of:
(a) locating one or more mammalian animals being treated with one or more cancer treatments;
(b) preparing a dosage in the range of between about 280 mg/kg/day and about 6300 mg/60 kg/day of said composition, wherein said composition comprises geranium oil and extractions from the root of Sophora tonkinesis; and
(c) delivering said dosage of said composition to said one or more mammalian animals being treated with said one or more cancer treatments.

61. The method of claim 60, wherein said dosage is about 350 mg/kg/day.

62. The method of claim 60, wherein said dosage is about 2100 mg/60 kg/day.

63. The method of claim 60, wherein said cancer treatment is 5-Fluorouracil.

64. A method for administering a composition comprising the steps of:
(a) locating one or more mammalian animals being treated or to be treated with one or more cancer treatments;
(b) determining a route of administering said composition to said one or more mammalian animals;
(c) determining a form of said composition to be administered to said one or more mammalian animals;
(d) obtaining a therapeutically effective dosage of said composition wherein said composition comprises geranium oil and extractions from the root of Sophora tonkinesis;
(e) placing said composition in an apparatus for administration;
(f) determining a time interval between separate administrations; and
(g) delivering said dosage of said composition to said one or more mammalian animals being treated or to be treated with said one more cancer treatments following said interval.

65. The method of claim 64, wherein said time interval is one to fourteen days.

66. The method of claim 64, wherein said time interval is within twenty-four hours or within forty-eight hours.

67. A composition comprising geranium oil and extractions from the root of Sophora plants.

68. The composition of claim 67, wherein said root of Sophora plants are root of Sophora alopecuroides.

69. The composition of claim 67, wherein said root of Sophora plants are root of Sophora moorcroftiana.

70. The composition of claim 67, wherein said root of Sophora plants are root of Euchresta strigillosa.

71. The method of claim 60, wherein said composition comprises the composition of claims 14, 15, 16, 17, or 18.

72. The method of claim 64, wherein said composition comprises the composition of claims 14, 15, 16, 17, or 18.
